(19) United States
(12) Patent Application Publication
(10) Pub. No.: US 2008/0215019 A1
(43) Pub. Date: Sep. 4, 2008

(54) Device For Treatment Of Wounds

(76) Inventor: Eugen Malamutmann, Dortmund (DE)

Correspondence Address: K & L GATES LLP, 535 SMITHFIELD STREET, PITTSBURGH, PA, 15222, US

(21) Appl. No.: 11/791,356

(22) PCT Filed: Nov. 22, 2005
(86) PCT No.: PCT/EP05/12494

§ 371 Date: Nov. 14, 2007

(30) Foreign Application Priority Data
Nov. 23, 2004 (DE) 10 2004 056 456.6

(51) Int. Cl. A61M 35/00

(52) U.S. Cl. 604/305

(57) ABSTRACT

A wound treatment device comprises a lower part (1) and an upper part (3), wherein the lower part is attached to the healthy skin in the area of a wound and completely surrounds the wound. Through the upper part (3) supply devices (7, 9) for treatment mediums are guided. The treatment mediums are discharged together with wound secretion by a discharge device (11). For exactly positioning the supply (7, 9) or delivery device (11) fixing means (10) guided through the upper part are employed.

The invention relates to a device for treatment of wounds in accordance with the preamble of claim 1.

The use of treatment mediums such as liquids, gases or else solids is well-targeted to accelerate the healing process of a wound. In order to keep the wound surface sterile and to exclude contamination of the environment it is necessary, however, to protect the wound area by a dressing or a cover. Moreover it is useful to get access to the wound surface without removing the dressing and/or the cover.

DE 28 09 828 A1 discloses a device for rinsing out wounds. It consists of a sheath connected to an edge, wherein either the sheath or the edge is provided with an inlet port and an outlet port for the rinsing liquid used.

Concerning the application of active principles in DE 197 22 075 C2 a device is disclosed in which a porous insert tightly covered by a film is applied to a wound. A liquid active principle is supplied and/or discharged via a feeding line and a draining line.

In EP 0 505 478 B1 a treatment system for wounds and other disturbances is disclosed. This treatment system consists of a flexible adaptable chamber comprising a bellows corrugation for receiving a treatment medium which can be sealed around the periphery of a wound. As an option, the system includes admitting means for introducing and removing treatment mediums, control means for treatment variables and surveying means for surveying wound conditions.

DE 196 81 649 T1 discloses a transportable therapeutic device for stimulating the healing of superficial wounds. This device comprises a casing including a suction pump and a reservoir for receiving liquid sucked off the wound by the pump. Moreover controlling means and indicators are disposed at the casing.

The device for treatment of wounds according to the invention is provided with a cover by which a chamber is formed above the wound. At least one functional means protruding into the chamber for treating the wound is arranged at the cover.

Said functional means consists of at least one manipulating means for treating the wound. According to an aspect of the invention, the manipulating means consists of a supply means for supplying treatment mediums and a discharge means for discharging existing mediums and wound secretion from the wound area again. Especially the supply means and the discharge means are in the form of supply and discharge tubes. This design permits, apart from the uniform treatment of the entire wound surface, a well-directed treatment of individual wound spots.

It is possible in this context to use multi-channel tubes so that through one tube at least two different treatment mediums can be supplied. Furthermore, through another channel also the discharge of the medium from the wound is possible. Liquid as well as gaseous or solid substances serve as treatment mediums, wherein any combination of said substances is possible.

It is equally possible to use other means such as, for instance, a surgical intervention means as manipulating means so as, for instance, to take swabs from the wound surface or to remove necrotic tissue.

In particular also electrodes, thermal elements, UV radiation elements or laser radiation elements can be provided as manipulating means.

In addition, the functional means may include an auxiliary means assisting the function of the manipulating means.

According to an aspect, the auxiliary means is a positioning element. With the aid of said positioning element it is possible to keep the manipulating means at a particular position inside the chamber.

The positioning elements may comprise an operating element rotatably supported in the cover which has an eccentric portion protruding into the chamber. Said eccentric portion is in mesh with a respective portion of the manipulating means provided in the chamber, wherein the respective portion of the manipulating means can thus be positioned as desired.

Additionally the positioning element can be manufactured of a plastic material. Consequently, it becomes possible by a plurality of positioning elements to arrange corresponding portions of the manipulating means exactly at particular positions inside the chamber. The positioning elements especially serve for arranging the supply and discharge means in the chamber in such a way that a well-directed treatment of a selected area of the wound surface is possible.

According to the invention, the chamber is formed by a lower part adapted to be permanently attached to the skin of a patient and at least one detachable upper part above the wound. The lower part is preferably made of soft material which adjusts to the shape of a body. The split configuration of the chamber formed in this way permits a direct access to the wound surface at any time, if necessary.

In the assembled state the interior of the chamber is sealed against the environment in a fluid-tight manner. The lower part can be attached to the healthy skin surrounding the wound especially by sewing, gluing or by other appropriate methods.

Preferably a film element is provided between the lower part and the healthy skin. Said film element is adapted to the shape of the wound so as to protect, on the one hand, the healthy skin against the treatment mediums and, on the other hand, to serve as sealing member.

In the upper part a plurality of passages adapted to be sealed in a fluid-tight manner are provided through which a plurality of different functional means can be guided.

The upper part can be made of a transparent material which permits a visual inspection of the interior of the chamber. Thus, it is possible, on the one hand, to check if the functional means are correctly arranged and it is possible, on the other hand, to visually inspect the surface of the wound itself. For the same purpose the upper part can be provided with flaps that are fluid-tight in a closed state.

In accordance with an aspect of the invention, the lower part is in the form of at least one inflatable sleeve and the upper part is formed of at least two members, with a substantially cylindrical chamber being formed. Said shape of the chamber permits to efficiently treat wounds in the area of arms and legs.

The chamber is particularly designed in the form of a bag for treating wounds at hands and feet. In order to avoid pressure sores, protective devices are provided at appropriate positions in the chamber.

It is especially also possible to connect an above-mentioned chamber for treating arms and legs at one end to an above-mentioned chamber for treating hands or feet, the sleeve being dispensed with in the area of the connecting portion. Thus, an elongated chamber is formed by which the treatment of wounds extending from the foot or the hand along the leg or arm, respectively, is possible.

According to another aspect, the lower part is formed of a continuous profile the ends of which are connected to a connecting member. The upper part is formed of rib elements and a film element. In the rib elements a plurality of openings adapted to be sealed fluid-tightly are provided through which the functional means are guided into the chamber. This design permits a very flexible adaptation to the shape of the wound.

According to a further aspect, the lower part and the upper part are integrally formed. For the treatment of wounds an integrally designed supply and discharge device is used. In this way, the treatment of small wound spots or skin disturbances such as, e.g., fistulae is possible.

The treatment fluids are delivered from a delivery device in the form of an end element of a supply means. The delivery device includes a plurality of chambers, wherein a first fluid is injected from a first chamber into a second fluid in a second chamber. The fluid mixture thus formed is then delivered to the outside.

Preferably the first fluid is a liquid and the second fluid is a gas, the liquid being injected into the gas and being atomized to form a mist. The mist is then delivered through delivery ports from the second chamber to the outside to the wound.

Hereinafter plural embodiments of the invention shall be illustrated in detail with reference to the Figures.

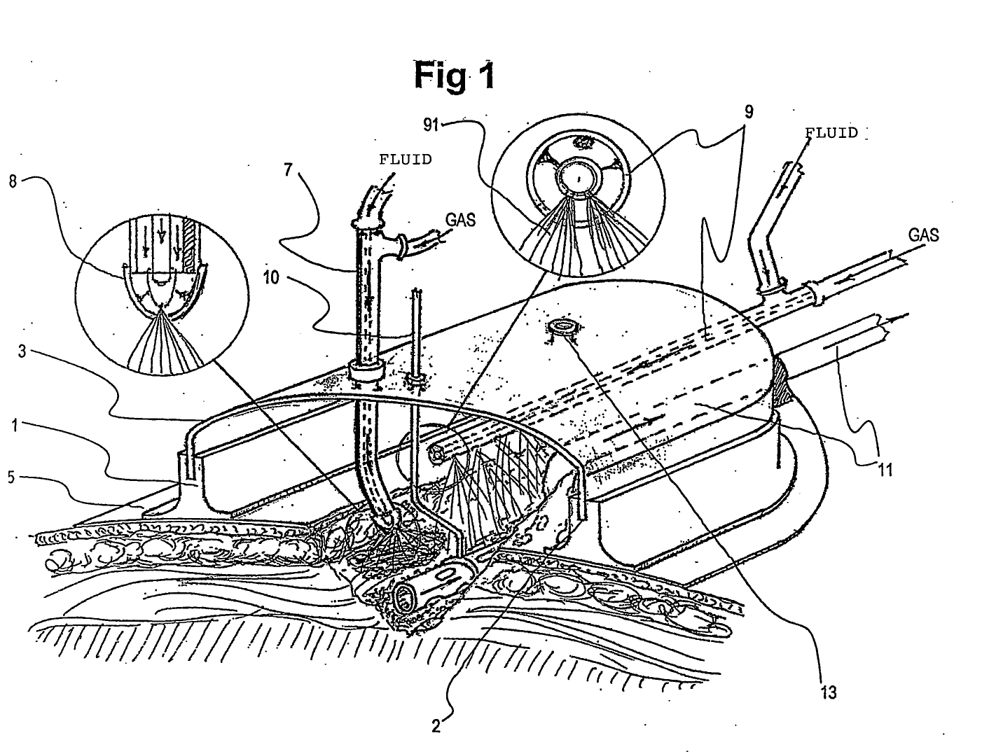

FIG. 1 shows a cutaway view of a wound cleansing chamber applied to a wound.

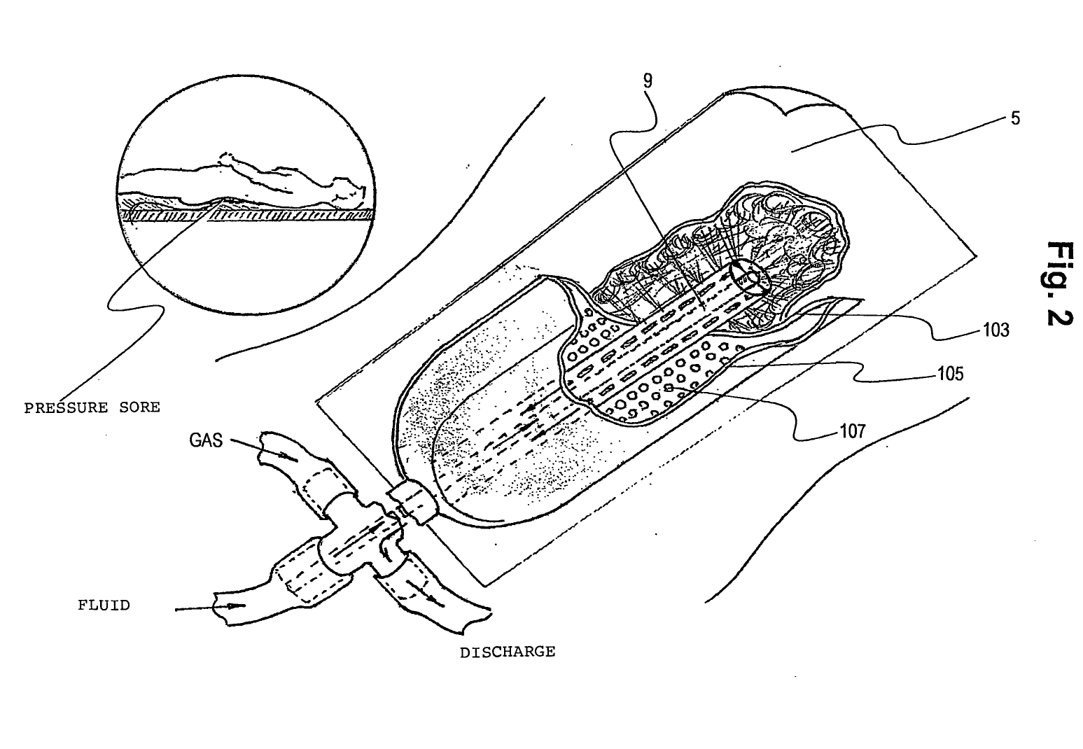

FIG. 2 shows a second embodiment of the wound cleansing chamber provided especially for use in the area of the sacrum (decubitus).

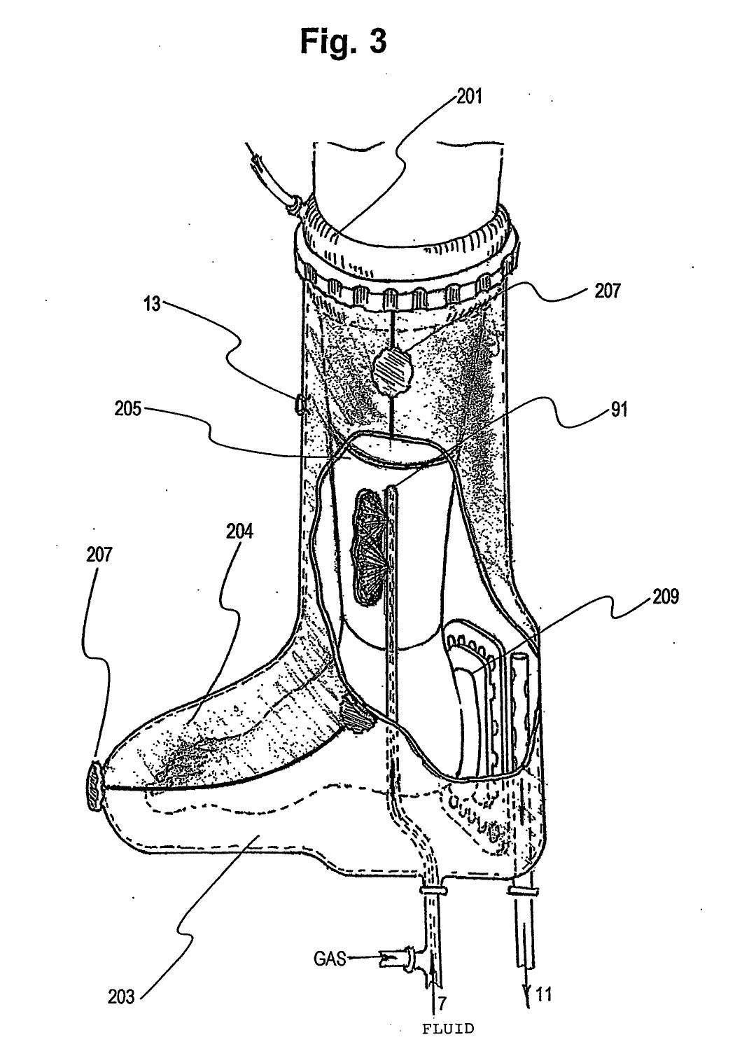

FIG. 3 shows a cutaway view of a chamber according to a third embodiment provided for use in the area of a foot.

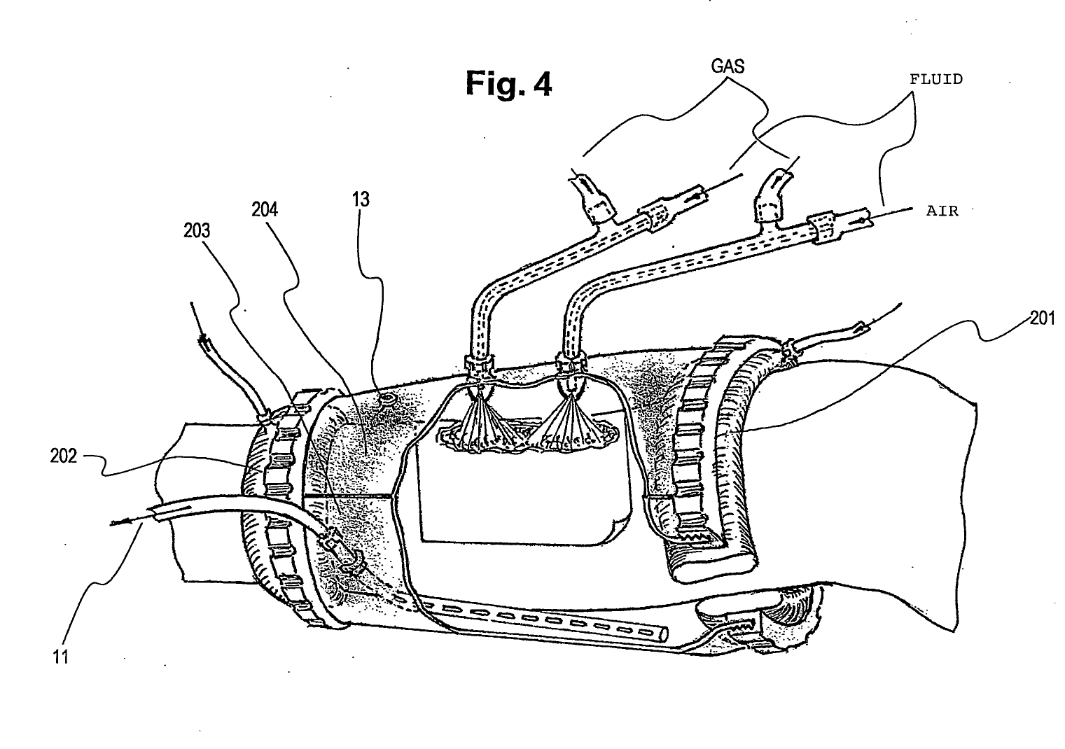

FIG. 4 shows a cutaway view of a chamber according to the third embodiment which is provided for use at an arm or a leg.

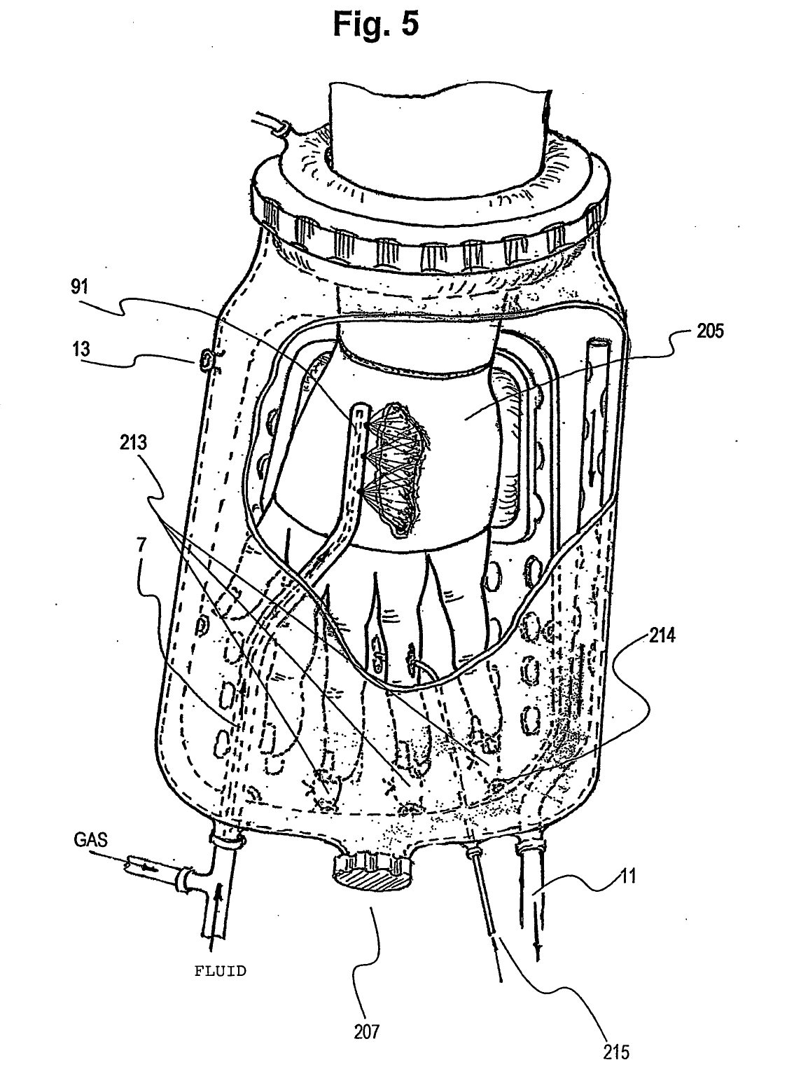

FIG. 5 shows a cutaway view of a chamber according to the third embodiment which is provided for use in the area of a hand.

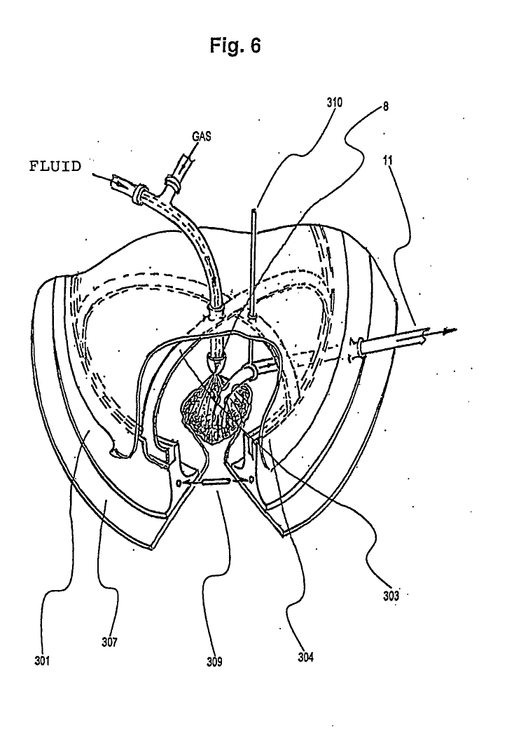

FIG. 6 shows a cutaway view of a chamber according to a fourth embodiment which is provided for punctual application with a fistula.

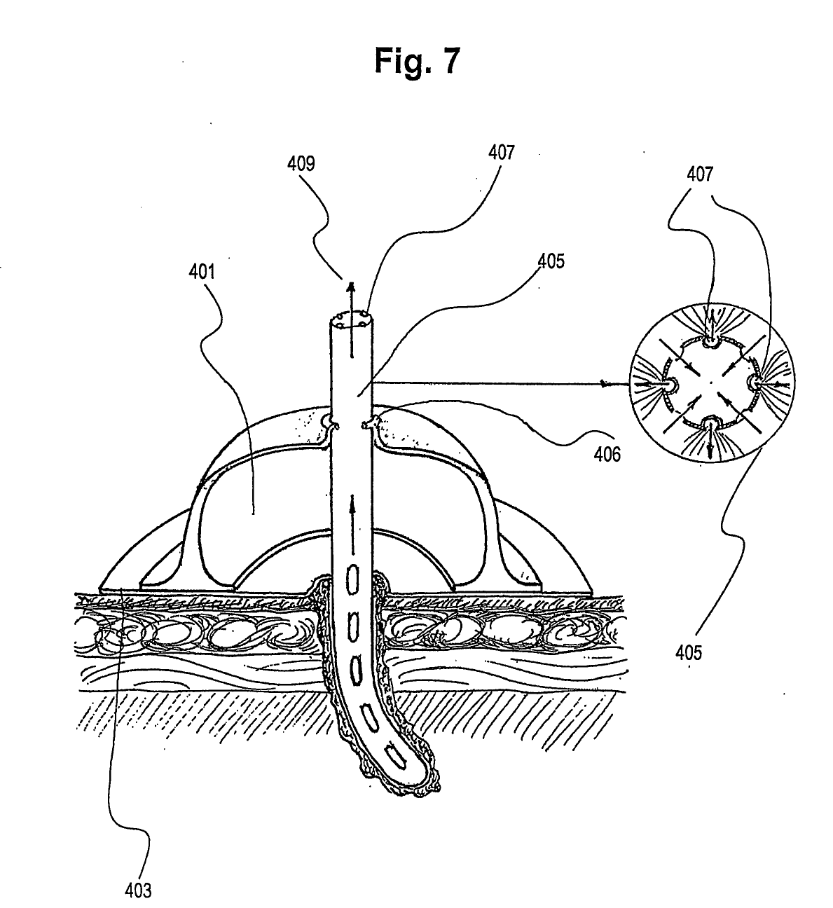

FIG. 7 shows a cutaway view of a chamber according to a fifth embodiment.

The basic structure of a wound treatment device according to the invention is evident from FIG. 1. A lower part 1 manufactured of a soft material consists of a wall portion and a fastening portion arranged beneath the wall portion and forms a chamber with the upper part 3. The lower part 1 is attached to the healthy skin of the patient in the area of the wound. For protecting the healthy skin a skin protective film 5 adapted to the shape of the wound is provided between the healthy skin and the underside of the lower part 1. The skin protective film 5 serves for protecting the healthy skin inside the chamber against the treatment mediums. For this purpose, the skin protective film 5 is adapted to the shape of the wound by means of an appropriate instrument, such as e.g. a pair of scissors. The lower part is attached by sutures not shown. Due to the soft material the lower part can optimally adjust to any body shape.

The upper part 3 is inserted in a groove 2 provided on the upper side of the lower part 1. In the upper part openings 13 are provided through which at least one supply tube 7, 9 serving as manipulating means is guided into the interior of the chamber.

According to the present embodiment, the supply tube 9 is a dual-channel tube through which the simultaneous supply of two different mediums, e.g. the supply of a liquid and of a gas, is permitted. The treatment mediums are in general sprayed through delivery ports 91 provided in the tube which are arranged at regular distances in a circumferential and longitudinal section of the tube.

For a punctual treatment of individual wound spots the treatment mediums can also be supplied through a nozzle 8 provided at the end of a supply tube 7. The openings 13 serve as manipulating openings for introducing further manipulating means into the chamber or for the supply or discharge of air. For the protection of the wound, on the one hand, and of the environment, on the other hand, a known air filter is provided in the openings formed for air supply and/or discharge.

Through an opening provided in the lower part 1 a discharge tube 11 likewise serving as manipulating means is guided as discharge means for the treatment mediums and wound secretion. The discharge tube 11 is kept in position by at least one positioning member 10 guided through the opening in the upper part 3 into the interior of the chamber, wherein the positioning member 10 has an eccentric portion and is rotatable from the outside of the chamber. It is possible in this way to adjust any position inside the chamber for the discharge tube 11. The positioning member 10 fulfills the function of auxiliary means and serves as functional means jointly with the supply and delivery tubes 7, 9.

FIG. 2 illustrates a second embodiment provided especially for treatment of wounds in the dorsal region. An inner wall 103 of an upper part having double walls is provided with an integrated supply tube/discharge tube 9.

In order to prevent the fluids to be discharged from accumulating in the wound area, the inner wall 103 of the upper part is provided with ports 107 through which the fluid can flow into the area between the two walls 103 and 105. From this area it is discharged through the third chamber of the tube 9.

FIG. 3 shows a third embodiment of the invention provided for use with wounds in the foot region. In this embodiment the lower part is an inflatable sleeve 201 to seal the entire circumference of the leg. The upper part 3 of this embodiment consists of two members 203 and 204 which are connected to each other and jointly with the sleeve 201 form a chamber adapted to the shape of the foot.

Auxiliary means 207 which moreover keep together the members 203, 204 along the connecting line thereof serve for mounting the supply tubes 7. As supply device 7 again a supply tube 7 is used. The discharge tube 11 provided in the calcanean region serves for the discharge of the respective mediums.

In order to avoid pressure sores gel cushions 209 are provided as protective means in the regions susceptible to pressure sores. Moreover, also in this case a skin protective film is applied for protection of the healthy skin.

In FIG. 4 an application of the third embodiment is shown with a wound in the area of the legs or arms. As lower part respective sleeves 201, 202 are used at the upper and at the lower end of the chamber. The supply and discharge of the respective mediums is carried out in accordance with the requirements for treating the wound by the already explained supply and discharge devices. The upper-part of said chamber consists of two members 203 and 204.

FIG. 5 shows the application of the third embodiment with a wound in the hand region. For fixing the fingers, fixing eyelets 214 and fixing points 213 are provided in the area of the finger tips. Otherwise the chamber shown in FIG. 5 corresponds to the chamber shown in FIG. 3 except for the size and the shape.

FIG. 6 illustrates a fourth embodiment of the present invention. In this embodiment the lower part is formed of a continuous profile 301 the ends of which are connected to a connecting element 309. The continuous profile 301 is attached to the skin by a skin protective film 307 provided therebetween. A delivery tube 11 forming the delivery device is guided through an opening in the continuous profile 301.

The upper part according to this embodiment is formed of rib elements 303 and a cover film 304. After attaching the continuous profile 301 to the skin, the skeleton of the upper part consisting of rib elements 303 is formed, wherein the ends of the rib elements 303 are arranged in a groove on the upper side of the continuous profile 301. In each rib element openings corresponding to those of the first embodiment are provided. The wound cleansing chamber according to this embodiment may cover the entire body.

This is of advantage especially when treating large-area burns.

After arranging the supply 7, discharge 11 and fixing devices 310 already explained by way of the first embodiment, the upper part is completed by disposing a cover film 304. According to the present embodiment, the film 304 is connected to the continuous profile 301 by gluing. After gluing the film the chamber fulfills the same functions as the chamber of the first embodiment.

A fifth embodiment of the invention is shown in FIG. 7. The chamber according to the fourth embodiment serves for the treatment of small wounds or defects, such as e.g. a fistula. According to this embodiment, the lower part and the upper part are designed as combined cover chamber 401.

A combined supply/discharge tube 405 is guided through an opening 406 in the cover chamber 401. The end of said tube 405 is inserted in the same up to the end of the fistula. After that, the cover chamber 401 is attached to the skin, as already described in the first embodiment. Through rinsing passages provided in the tube 405 a treatment medium is supplied to the fistula and is then discharged again together with wound secretion through a delivery passage 409.

A control unit can be combined with all embodiments of the invention. By said control unit it is possible to program the fluid supply and discharge as well as the amount of treatment mediums and to adjust the internal pressure of the chamber. In particular the digital control unit is also adapted to control the gas and liquid volume and the sucking of the fluids.

1-16. (canceled)

17. A device for treatment of wounds comprising a cover for forming a chamber above a wound at which at least one functional means protruding into the chamber is arranged for treating the wound, wherein the functional means includes at least one manipulating means, wherein the chamber is formed by at least one lower part attachable to the skin and at least one upper part, wherein the upper part is designed to be detachable from the lower part, and at least one manipulating means of the functional means is an electrode, and that a skin protective film is provided to be located between the skin and the lower part, and an opening provided with an air filter is arranged in the upper part.

18. The device according to claim 17, wherein another manipulating means is in the form of a surgical intervention means and/or a supply means and a discharge means.

19. The device according to claim 18, characterized in that the supply means and the delivery means are in the form of supply and/or discharge tubes for supplying a medium into the chamber and/or discharging a medium from the chamber.

20. The device according to claim 19, wherein the supply and/or delivery tubes are multi-channel tubes.

21. A device according to claim 20, wherein the supply and delivery tubes are formed in a line in one piece.

22. A device according to claim 17, wherein the functional means additionally includes at least one auxiliary means.

23. A device according to claim 22, wherein the auxiliary means is a positioning element for positioning the manipulating means.

24. The device according to claim 23, wherein the positioning element has an operating element rotatably supported on the cover and comprising an eccentric portion protruding into the chamber.

25. The device according to claim 23, wherein the positioning element is made of plastic material.

26. The device according to claim 17, wherein the lower part consists of at least one sleeve and the upper part is substantially cylindrical and is formed of at least two pieces.

27. The device according to claim 26, wherein the upper part is in the form of a bag at the opening of which the sleeve is disposed.

28. The device according to claim 26, wherein the upper part has the shape of an open cylinder at the two ends of which a respective sleeve is arranged.

29. The device according to claim 26, wherein the sleeve is inflatable.

30. The device according to claim 17, wherein the lower part is made of a continuous profile and the upper part consists of a film element supported on reinforcing elements.

31. The device according to claim 17, wherein the upper part is double-walled comprising an inner wall and an outer wall and the inner wall has openings.
